SEQUENCE LISTING (2 sequences)

SEQ ID NO: 1 - DNA, 31 bp, Artificial Sequence
  Primer
aaaggatccaaaccacatatcgataattatc

SEQ ID NO: 2 - DNA, 31 bp, Artificial Sequence
  Primer
aaggatccttatttgacttctgtagctacaa

(19) United States
(12) Patent Application Publication
(10) Pub. No.: US 2004/0230033 A1
(43) Pub. Date: Nov. 18, 2004

(54) Substrate-derived affinity labels, and uses thereof

(76) Inventor: Brian Walker, Belfast (GB)

Correspondence Address: DRINKER BIDDLE & REATH, ONE LOGAN SQUARE, 18TH AND CHERRY STREETS, PHILADELPHIA, PA, 19103-6996, US

(21) Appl. No.: 10/437,376

(22) Filed: May 12, 2003

(51) Int. Cl. C07K 5/06

(52) U.S. Cl. 530/330; 530/331

(57) ABSTRACT

A modified peptide comprises the formula:

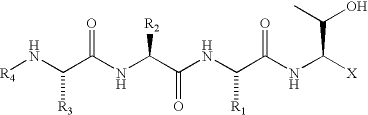

wherein R₁ is a threonine side chain, R₂ is a side chain of any amino acid, R₃ is a side chain of proline, R₄ is an acetyl group or an N-protecting group, Y is a halogen, R₅ is a natural amino acid side chain, and wherein X is an electrophilic group having an ability to undergo nucleophilic attack with active site cysteine residues in cysteine proteinase enzymes.

### FIELD OF THE INVENTION

The invention relates to modified peptides and their use as substrate-derived affinity labels. In particular, the invention relates to the use of the affinity labels in detecting functionally active cysteinal protease-transpeptidase sortase (SrtA) and SrtA-like proteins.

### BACKGROUND OF THE INVENTION

In Gram-positive pathogens, the cell surface is a complex milieu of proteins that play a pivotal role in adhesion to human tissues, as well as the evasion of host-immune responses [1,2]. A number of important virulence factors, such as Protein A, fibronectin-binding proteins A and B and collagen-binding protein are covalently attached to the bacterial cell wall by a pathway that is ubiquitous amongst Gram-positive organisms. These proteins are characterized by the presence of a distinctive C-terminal ‘sorting sequence’ composed of three different elements: a charged tail, a preceding hydrophobic sequence and a conserved-Leu-Pro-Xaa-Thr-Gly-motif (where Xaa represents any amino acid) [3-5]. An elegant series of experiments by Schneewind et al. [6-9] have now characterized the molecular events involved in the presentation of Leu-Pro-Xaa-Thr-Gly-proteins on the surface of Gram-positive organisms. They identified, cloned and sequenced a cytoplasmic membrane-bound cysteine protease-transpeptidase responsible for the cleavage and covalent linkage of proteins containing the conserved -Leu-Pro-Xaa-Thr-Gly-sequence motif. This constitutively expressed enzyme, known as staphylococcal surface protein sorting A (SrtA), contains a single active-site cysteine residue (Cys¹¹⁴), which catalyses (via nucleophilic attack) a highly specific cleavage of the scissile Thr-Gly peptide bond in -Leu-Pro-Xaa-Thr-Gly-. The thiol-acyl enzyme intermediate formed between the thiol group of Cys¹¹⁴ and the carbonyl group of Thr is subsequently linked, via an amide bond, to pentaglycine cross-bridges in the bacterial cell wall [10]. Very recently [11], Schneewind et al. identified a second sortase species (SrtB) in Staphylococcus aureus. This protease, which also appears to employ an active-site cysteine residue for catalysis, has a different subsite specificity from SrtA and cleaves surface proteins at an -Asn-Pro-Gln-Thr-Asn-signal sequence, before anchoring the polypeptide to the cell-wall envelope. The SrtB protease, which is transcribed from an iron-regulated operon, appears dedicated to the acquisition of iron from the host environment, which is important for the pathogenesis of S. aureus infections. Biological studies have shown that knockout mutation of the SrtA gene in S. aureus inhibits the organism's adherence to IgG, fibrinogen and fibronectin, and greatly reduces the capacity of the pathogen to establish an acute infection in mice [12]. In addition, inactivation of the SrtA gene in the human commensal bacterium Streptococcus gordonii significantly compromises the ability of the organism to colonize the oral mucosa in mice [13]. Studies with an SrtB mutant strain of S. aureus have revealed no significant reduction in pathogenicity during the early stages of infection in mice [11]. However, over a period of time, the level of infection in the animals injected with the mutant strain was found to decrease significantly when compared with a wild-type strain of S. aureus. This suggests that SrtB does not influence the initial establishment of infection, but is required for persistence of the pathogen within infected tissues. Therefore both SrtA and SrtB are important virulence factors, but they appear to play different roles in the infection process.

It is clear that, in an era of growing antibiotic resistance, both SrtA and SrtB may prove exciting new targets for the development of anti-staphylococcal drugs, or even broad-spectrum agents against Gram-positive pathogens.

### SUMMARY OF THE INVENTION

According to the invention, there is provided a modified peptide comprising the formula:

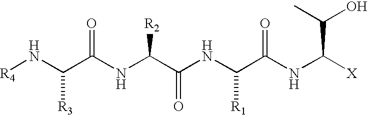

wherein R₁ is a threonine side chain, R₂ is a side chain of any amino acid, R₃ is a side chain of proline, R₄ is an acetyl group or an N-protecting group, Y is a halogen, R₅ is a natural amino acid side chain, and wherein X is an electrophilic group having an ability to undergo nucleophilic attack with active site cysteine residues in cysteine proteinase enzymes.

In a preferred embodiment of the invention, X is a substituent selected from the group comprising:

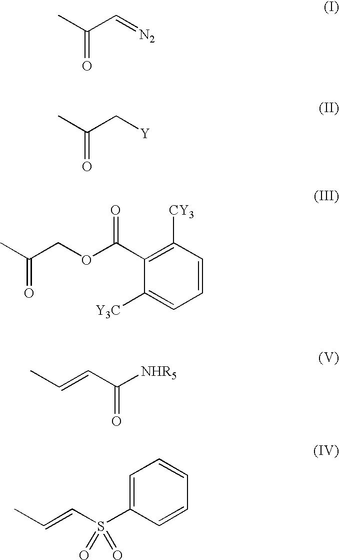

In one embodiment, the modified peptide according to the invention includes an assayable label. Generally, the label is biotin. In one embodiment, the N-protecting group comprises an assayable label linked to a spacer group. Typically, the spacer group is an aminohexanoyl group. Other suitable spacer groups will be known to those skilled in the field of peptide chemistry. In a particularly preferred embodiment of the invention, R4 is biotin-bis-aminohexanoic acid

In an alternative embodiment of the invention, the N-protecting group is a benzyloxycarbonyl group (Cbz).

In a particularly preferred embodiment of the invention, R₂ is a side chain of alanine.

In substituent III above, the halogen will generally comprises fluorine. Likewise, in substituent II above, the halogen will generally comprise chlorine.

The invention also relates to a modified peptide consisting of the molecular structure (I), (II), or (III) of FIG. 4.

The invention also relates to a use of a modified peptide according to the invention as an inhibitor of cysteine peptidase enzymes, especially sortase enzymes including staphylococcal surface protein sorting A (SrtA) and SrtA-like proteins. In this specification, the term “SrtA-like enzymes” should be understood as meaning proteins which catalyse a highly specific cleavage of a scissile threonine-glycine amide bond of a conserved-LPXTG-recognition motif.

The invention also relates to a medicament comprising a modified peptide according to the invention.

The invention also relates to a medicament comprising a modified peptide selected from the molecular structures (I), (II) or (III) of FIG. 4.

The invention also relates to a pharmaceutical composition comprising a pharmaceutically acceptable carrier and one or more modified peptides according to the invention.

The invention also relates to an anti-bacterial agent comprising a modified peptide according to the invention.

The invention also relates to an invasive medical device such as a stent, a catheter, or a prosthesis, which includes a modified peptide according to the invention. Typically, the modified peptide is exposed on the surface of the medical device.

The invention also relates to a method of treating a bacterial infection in an individual comprising the step of administering to the individual an effective amount of a modified peptide according to the invention. Suitably, the modified peptide is selected from the molecular structures (I), (II) or (III) of FIG. 4.

The invention also relates to the use of a modified peptide according to the invention to detect functionally active serine protease enzymes, especially sortase enzymes such as SrtA and SrtA-like proteins, in materials. Accordingly, a method for detecting the presence of functionally active serine protease enzyme in a material comprises contacting the material with a modified peptide according to the invention.

The material in which the functionally active serine protease enzymes are detected in one embodiment is a biological extract or sample. Advantageously, the sample or extract is a bacterial lysate, the preparation of which is described in the herein Examples, below.

The invention also relates to a method of purifying functionally active SrtA or SrtA-like enzymes in a biological extract, which method comprises the steps of:

incubating the biological extract with a labelled modified peptide according to the invention to allow the labelled modified peptide bind to any SrtA or SrtA-like enzymes present in the extract; and

purifying any thus labelled enzymes.

Typically, the purification step comprises affinity chromatography. In this regard, the label is suitably biotin, and the chromatography column will generally comprise streptavidin. In one embodiment of the invention, the method includes the further steps of mass spectral analysis and/or amino acid sequencing of the or each purified enzyme.

The invention also relates to a method of detecting functionally active SrtA or SrtA-like enzymes in a biological extract, which method comprises the steps of:

incubating the biological extract with a labelled modified peptide according to the invention to allow the labelled modified peptide bind to the or each SrtA or SrtA-like enzyme; and

treating the incubated biological extract to separate out proteins in the extract; and

carrying out a Western-blot analysis of the separated proteins.

Typically, the proteins in the extract are separated using PAGE.

### BRIEF DESCRIPTION OF FIGURES

The invention will be more clearly understood from the following description of some examples thereof, given by the way of example only, with reference to the accompanying Figures in which:

FIGS. 1 to 3 illustrate schemes for synthesising modified peptides according to the invention;

FIG. 4 illustrates the molecular structures of the substrate-derived SrtA_{Δn} inhibitors in which (I) corresponds to the peptidyl-diazomethane inhibitor Cbz-Leu-Pro-Ala-Thr-CHN₂, (II) corresponds to the peptidyl chlormethane inhibitor Cbz-Leu-Pro-Ala-Thr-CH₂Cl, and (III) corresponds to the biotin-peptiedyl-diazomethane inhibitor biotin Ahx-Leu-Pro-Ala-Thr-CHN₂;

FIG. 5 illustrates the strategy for solution solid phase synthesis of the substrate-derived SrtA_{Δn} inhibitors (Synthetic conditions: i, 20% (v/v) piperidine-dimethylformamide for 15 min; ii, Fmoc-Ala-OH/HBTU/DIPEA (di-isopropyl ethylamine; 1:1:2, by vol.) for 30 min; iii, repeat step i; iv, Fmoc-Pro-OH/HBTU/DIPEA (1:1:2, by vol.) for 30 min; v, repeat step i; vi, for R=Cbz, Cbz-Leu-OH/HBTU/D1 PEA (1:1:2, by vol.), 30 min; for R=biotinyl-Ahx-, Fmoc-Leu-OH/H BTU/D1 PEA (1:1:2, by vol.), 30 min, then repeat step i, followed by biotin/HBTU/DIPEA (1:1:2, by vol.), 2×30 min; vii, trifluoroacetic acid/double-distilled water/tri-isopropyl silane (95:2.5:2.5, by vol.), 90 min at 0° C.; viii, isobutyl chloroformate (1.2 eq.), N-methyl morpholine (1.2 eq.), 15 min at 0° C., then add ethereal diazomethane (6.0 eq.), stir at room temperature, overnight; ix, ethereal HCI (6.0 eq.), 3 min at 0° C.

FIG. 6 illustrates the mechanism of inhibition by the substrate-derived peptidyl diazomethane (I) and peptidyl-chloromethane (II) SrtA_{Δn} inhibitors;

FIG. 7 is a graph of the reciprocal of the apparent second-order rate constant A for inactivation versus inhibitor concentration [I] (Hydrolysis of the internally quenched substrate babcyl-Gln-Ala-Leu-Pro-Glu-Thr-Gly-Glu-Edans was performed in the presence of five different concentrations (25, 50, 100, 150 and 200 μMember) of inhibitor II of FIG. 4; and

FIG. 8 is a Western Blot analysis of recombinant and wild-type SrtA after affinity labelling with the biotin-Ahx-Leu-Pro-Ala-Thr- CHN₂ (III)

### DETAILED DESCRIPTION OF THE INVENTION

Materials

All protected amino acids, Wang Resin and coupling reagents used for peptide synthesis were purchased from CalbiochemNovabiochem (Nottingham, U.K.). All solvents for peptide synthesis were obtained from Applied Biosystems (Warrington, Cheshire, U.K.). S. aureus (Oxford strain) was from the A.T.C.C. 9144 (Manassas, Va., U.S.A.). Luria-Bertani (LB) broth was purchased from Oxoid (Basingstoke, U.K.). The pET-3d expression system, and the antibiotics ampicillin and chloramphenicol were purchased from Novagen (Madison, Wis., U.S.A.). Nit+nitrilotriacetate (Ni-NTA)-agarose was purchased from Qiagen (Crawley, West Sussex, U.K.). Biotin, BSA, the substrates 5-bromo-4-chloroindol-3-yl phosphate and Nitro Blue Tetrazolium, isopropyl P-D-thiogalactoside and goat anti-rabbit IgG alkaline phosphatase were from Sigma-Aldrich (Poole, Dorset, U.K.). Novex® SDS/PAGE gels were from Invitrogen (Groningen, The Netherlands), whereas streptavidin alkaline phosphatase was from Vector Laboratories (Peterborough, U.K.). Rabbit antiserum raised against recombinant sortase SrtA_{ΔN} was a gift from Fusion Antibodies Ltd. (Belfast, U.K.).

Synthesis of Substrate-derived Inhibitors

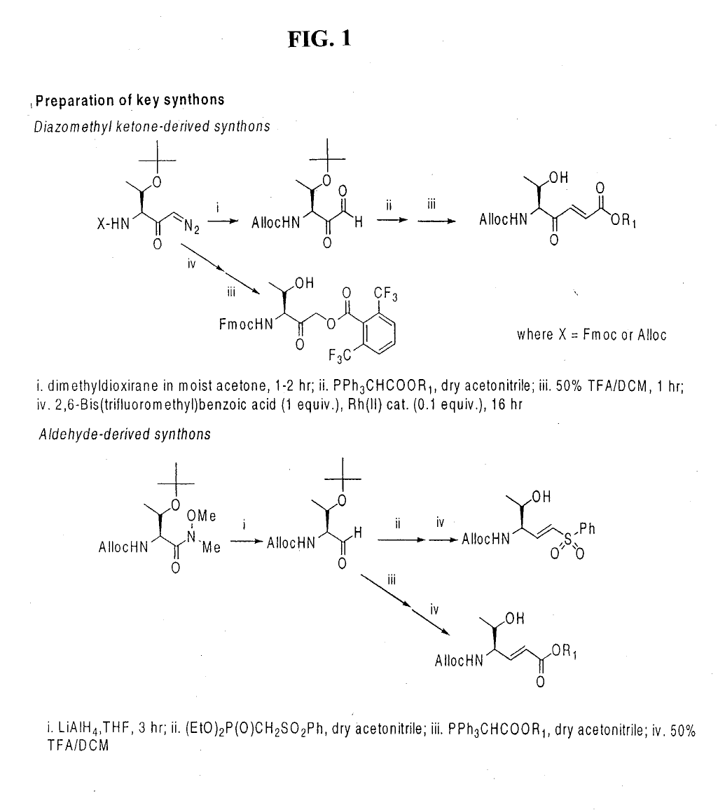

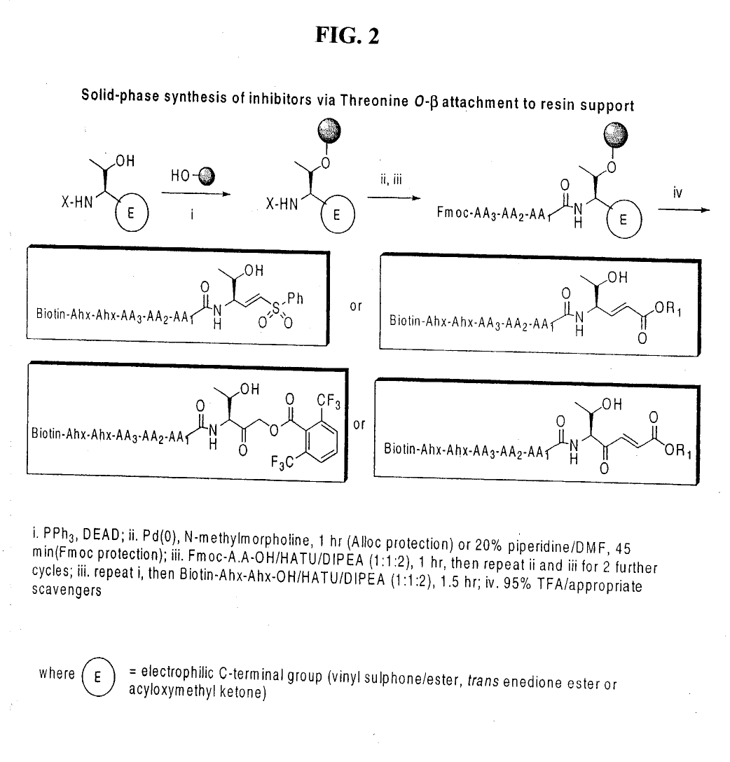

FIGS. 1 to 3 outline the detailed synthetic approaches for the preparation of inhibitors which utilise a series of solid- and solution-phase synthetic protocols. Essentially, N-α-protected threonine derivatives incorporating C-terminal electrophilic functions are incorporated onto solid supports, either via the C-terminal functionality (vinyl and trans enedione acids) or via the β-hydroxyl functionality (vinyl sulphone, diazomethyl ketone, chioromethyl ketone, acyloxymethyl ketone). Peptide targeting sequences are synthesised using standard orthogonal Fmoc-/tBu strategies. We have utilized these sequences (incorporating either an N-terminal acetyl- or biotinyl-(aminohexanoyl)₂-group) as starting templates and subsequently synthesised peptide fragments of the form Leu-Pro-Ala-Thr- with positional scanning at each of the P2-P4 positions. This affords a considerable degree of molecular diversity within the final library of biotinylated affinity labels and allow the exploration of the importance of each of these 3 regions in individually disclosed sortase-like species. The synthetic schemes reflect the divergent nature of the synthetic protocols. Essentially, the classes of inhibitors are prepared from orthogonally protected N-α-Fmoc-threonine diazomethyl ketone or aldehyde starting synthons. N-α-protecting group strategies are dependent on application of the synthons in divergent synthetic protocols. Thus, those inhibitors incorporating the vinyl sulphone and amides employ N-α-allyoxycarbonyl (Alloc)-protected threonine as the starting synthons. This approach facilitates application to the Wittig olefination reaction, which would affect 9-fluorenylmethoxycarbonyl (Fmoc) protecting groups which will be used for all other syntheses.

Diazomethyl Ketone-based Starting Synthon

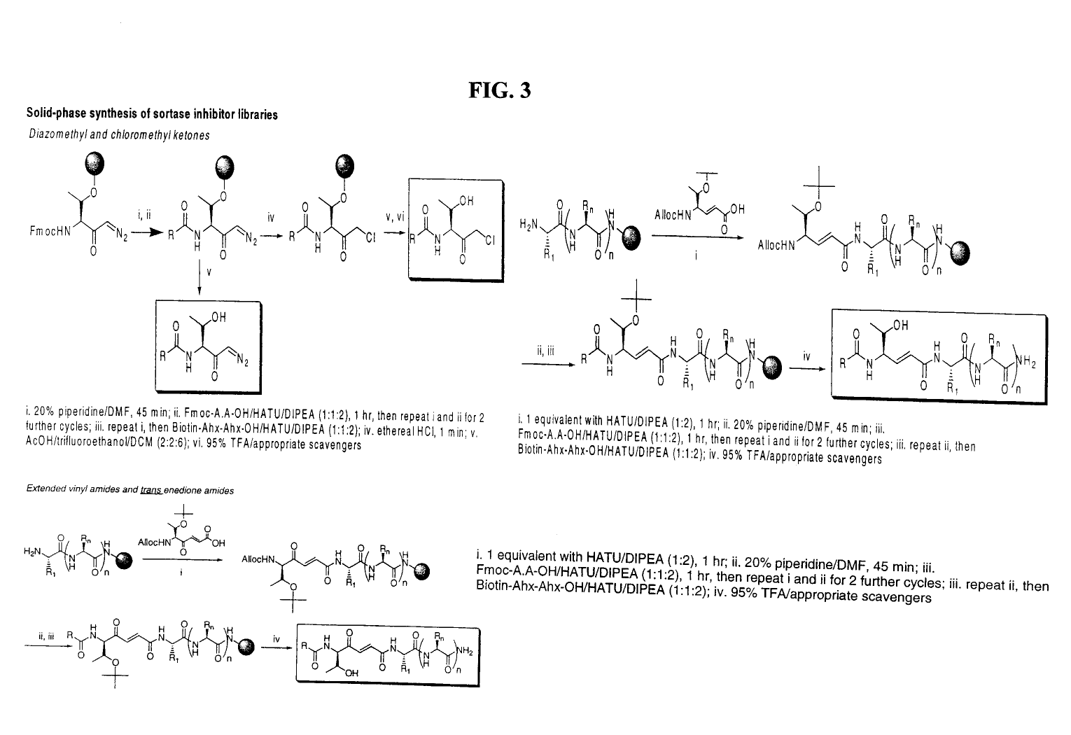

N-α Alloc-Thr(OBut)-OH is converted, via an unsymmetrical anhydride intermediate, into the corresponding diazomethyl ketone (Alloc-Thr(OBut)-CHN₂). Treatment with dimethyl dioxirane in moist acetone affords the α-keto-β-aldehyde intermediate in quantitative yield, which readily undergoes a Wittig olefination type reaction with a series of carboxyalkyl triphenylphosphoranes to yield trans enedione esters (14). The trans enedione methyl ester will be hydrolysed (1M LiOH/THF) to afford the free trans enedione acid, which will be employed for the synthesis of trans enedione amides (FIG. 3). In the presence of soluble rhodium (II) catalysts, diazomethyl ketones are also known to undergo insertion reactions with carboxylates to yield acyloxymethyl ketones (15). This approach is exploited for the synthesis of the orthogonally protected bis 2,6(trifluoromethyl)benzyloxymethyl ketone analogue of threonine. Synthesis of the N-protected-threonine-based starting synthons is accomplished in solution, after coupling to 2-chlorotrityl chloride, peptide sequences are elaborated on an Advanced Chemtech Vantage™ automated synthesiser employing standard solid-phase synthetic protocols.

Aldehyde-derived C-terminal Electrophiles

N-α-Alloc-Thr(OBut)-OH is converted to its corresponding aldehyde derivative via LiAlH₄-mediated reduction of the corresponding Weinreb amide (Alloc-Thr-(OBut)-N(MeO)Me). This is readily converted via Wittig olefination type reactions into either vinyl sulphone (16) or vinyl esters (17). The C-terminal trans enedione ester, acyloxymethyl ketone, vinyl sulphone and vinyl ester analogues of threonine, described above, are generated using standard solution-phase methodologies. Following removal of their 0-butyl ether protecting group (50% TFA/DCM), each derivative is attached to a HMP- or SASRIN resin-based solid support. This is achieved by a Mitsunobu reaction in each instance. Following attachment to the solid-phase, peptidyl derivatives of each warhead are synthesised using standard orthogonal Fmoc/tBu protocols (18). In each instance, after completion of synthesis, inhibitors are cleaved from the solid support via acidolytic cleavage (95% TFA/appropriate scavengers). Newly generated compounds are purified by reversed phase HPLC and characterized by ¹H NMR and electrospray mass spectral analysis

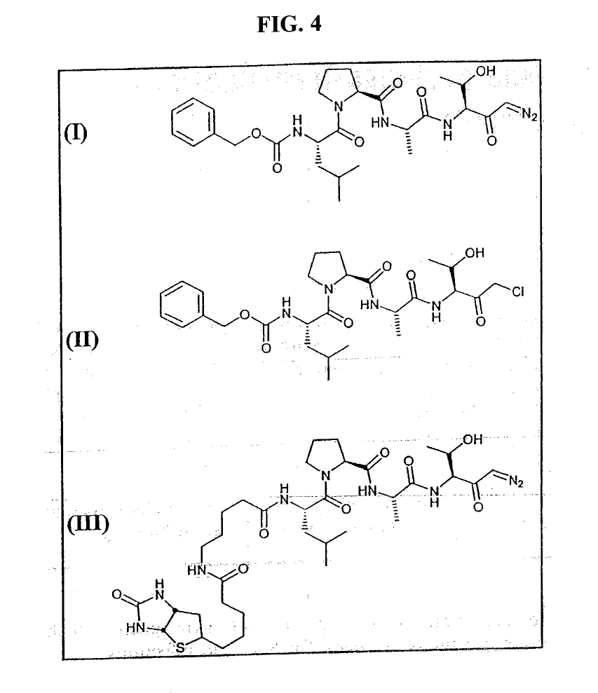

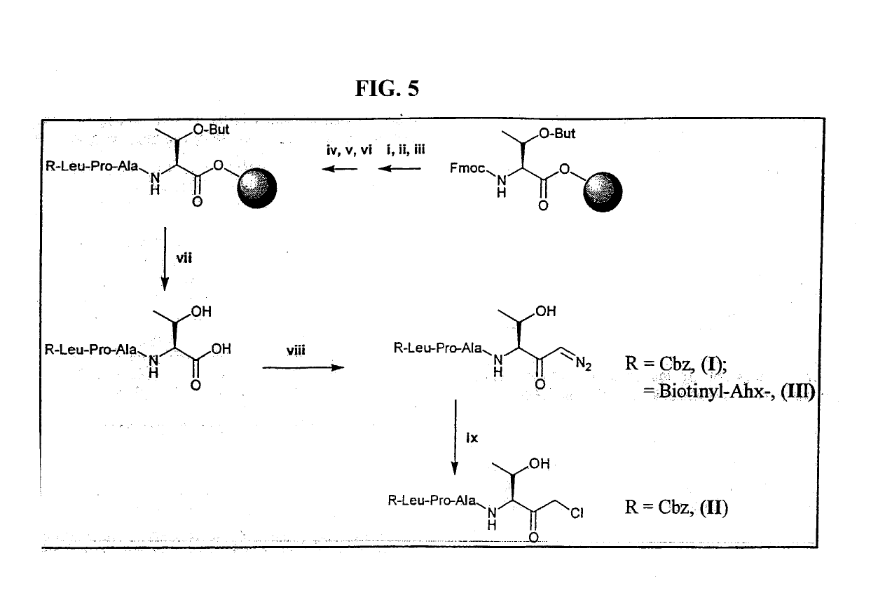

The substrate-derived inhibitor sequences illustrated in FIG. 4 were synthesized using a combination of solid-phase and solution ethodologies previously reported (FIG. 5; [19-21]). In essence, the Leu-Pro-Ala-Thr-portion of each inhibitor was synthesized (0.5 mmol scale) using standard solid-phase synthesis protocols on acid-sensitive Wang resin, previously derivatized with fluoren-9-ylmethoxycarbonyl (Fmoc)-Thr(OBut)-OH as the first amino acid [18]. Each subsequent amino acid was incorporated via single 2-(1H-benzotriazol-1-yl)-1,1,3,3tetramethyluronium hexafluorophosphate (HBTU)-mediated couplings. Both alanine and proline residues were incorporated as their N-a-Fmoc derivatives. For inhibitor sequences I and II, the common leucine residue was incorporated as an N-protected benzyloxycarbonyl (Cbz) derivative. For the biotinylated sequence III, the leucine residue was incorporated as an N-a-Fmoc derivative, as was the aminohexanoic linker residue. Biotin was incorporated into the sequence using a double-coupling step. On completion of each synthesis, the peptides were cleaved from the solid support by treatment with a 10 ml solution of 95% (v/v) trifluoroacetic acid containing 2.5% (v/v) double-distilled water and 2.5% (v/v) tri-isopropyl silane. Each cleavage reaction was concentrated to approx. 0.5 ml by evaporation under reduced pressure. The peptide product was then precipitated by dilution (1:20) with chilled diethyl ether and isolated by centrifugation (2000 g for 10 min). The products (yields of 80%) were dried in vacuo, for 12 h, before the next stage of the synthesis. The peptides (obtained as their C-terminal free acids) were then activated, via a mixed carbonic anhydride, and converted into their respective diazomethanes by reaction with ethereal diazomethane [19]. The peptidyl-chloromethane inhibitor (II) was prepared (in quantitative yield) by treating a sample (0.1 mmol) of inhibitor I with a 10 ml solution of anhydrous ethereal HCI, for 15 min at 0° C. [23]. The purity and identity of each product was confirmed by reverse-phase HPLC and matrix-assisted laser-desorption ionization-time-of-flight (MALDI-TOF)-MS.

PCR Cloning, Expression and Purification of Recombianant SrtA_{ΔN}

SrtA is believed to be a membrane-associated protease, with an N-terminal membrane anchor. To facilitate purification and solubility, a recombinant form of sortase (SrtA_{ΔN}) has previously been described and used in which the N-terminal membrane anchor segment of the enzyme (residues 2-25) was replaced with a hexahistidine (His₆) sequence [8,9,12]. In the present work, a similar strategy was adopted to create a construct for SrtA_{ΔN} by replacing the N-terminal T7 tag in a pET-3d expression vector with a His₆ tag. The region of the SrtA gene lacking the N-terminal membrane domain was then PCR-amplified from the genomic DNA of S. aureus (Oxford strain) using the primers orf5N-ds-B (5′-AAAGGATCCAAACCACATATCGATAATTATC-3′) (SEQ ID NO:1) and orf5C-B (5′-AAGGATCCTTATTTGACTTCTGTAGCTACAA-3′) (SEQ ID NO:2)[8], and the resulting amplicon was cloned into the expression vector at a unique BamHI restriction site. Competent Escherichia coli cells (JM109) were then transformed and selected on LB agar plates containing ampicillin (100, ug/ml). After selection and verification of positive clones by dideoxynucleotide DNA sequencing, the vector was transformed into the E. coli expression strain HMS174(DE3)pLysS. Transformed bacteria were propagated at 37° C. in LB broth containing ampicillin (100, ug/ml) and chloroamphenicol (35, ug/ml), and the expression was induced with 1 mM isopropyl/7-D-thiogalactoside. Cultures were harvested after a 4 h post-induction incubation at 37° C., and the recombinant enzyme was purified on a Ni-NTAagarose column as described previously [8]. Confirmation of SrtA_{ΔN} was achieved using a monoclonal antibody to the His₆ tag (Sigma-Aldrich) and ion-trap MS on peptides recovered from “in-gel” tryptic digests of the recombinant protein

Kinetic Analysis of Inhibitors Cbz-Leu-Pro-Ala-Thr-CHN_{z} (I) and Cbz-Leu-Pro-Ala-Thr-CH_{z}CI (II)

Using methods described previously [8,9], SrtA_{ΔN} activity was measured with the internally quenched substrate 4([4-(dimethylamino)phenyl]azo)-benzoyl(Dabcyl)-Gln-Ala-Leu-Pro-Glu-Thr-Gly-Glu-Glu-[(2-aminoethyl)-amino]napthalenel-sulphonyl (Edans) (prepared by standard Fmoc solid-phase synthesis) on a CytoFluor 4000® multi-well fluorimeter (PerSeptive Biosystems, Foster City, Calif., U.S.A.). All SrtA_{AN} assays were performed at 37° C. (in triplicate) in 50 mM Tris/HCI (pH 7.5) containing 150 mM NaCl, 5 mM CaCl2, 5 mM NH,-Gly₃ and 5 mM dithiothreitol (SrtA buffer). Wells contained approx. 10 pM of purified SrtA_{oN} and 50 pM of substrate, in a final volume of 200, ul. The Kₘ and k_{cat} values for the SrtA_{ΔN}-catalysed cleavage of the internally quenched sub-strate were calculated by fitting the data points directly into the Michaelis-Menten equation for substrate hydrolysis, using GraFit® software (Erithacus Software, Horley, Surrey, U.K.).

Affinity Labelling of Recombinant and Wild-type SrtA with Biotin-Ahx (Aminohexanoyl)-Leu-Pro-Ala-Thr-CHN_{z} (III)

For the detection and disclosure of SrtA_{ΔN} with biotin-Ahx-LeuPro-Ala-Thr-CHN₂ (III), crude lysates of transfected E. coli cells (freshly induced) were prepared, followed by purification of the recombinant enzyme on a Ni-NTA-agarose column as previously described [8]. For the disclosure of wild-type SrtA in S. aureus, a 50 ml overnight culture of the organism in LB broth was prepared. The bacterial cells were centrifuged at 3000 g for 15 min and the resulting pellet was washed in 5 ml of SrtA buffer. The washed bacteria were then resuspended in 1 ml of SrtA buffer containing 0.1 g of glass beads. The suspension was vortex-mixed continuously for 5 min followed by centrifugation at 1500 g for 15 min to remove the beads and unbroken cells. To 100 pl of purified SrtA_{ΔN} and the crude bacterial preparations, the inhibitor biotin-Ahx-Leu-Pro-Ala-Thr-CHN₂ (III) was added (final concentration 50 pM) before incubation at 37° C. for 30 min.

To detect the affinity-labelled recombinant and wild-type SrtA, samples were analysed by Western-blot analysis [19,24-26]. Briefly, samples were treated with denaturing treatment buffer and the proteins separated by SDS/PAGE on 4-20% (w/v) linear gradient gels, followed by semi-dry transfer to a nitrocellulose membrane (Schleicher and Schiill, Dassel, Germany). After transfer, non-specific binding sites were quenched with a 3% (w/v) solution of BSA in 20 mM Tris/HCI (pH 7.4), containing 150 mM NaCl. To detect the biotin group, the membrane was incubated with a streptavidin-alkaline phosphatase conjugate (1:500). To confirm the identity of affinity-labeled recombinant and wild-type SrtA, the expressed protein and crude S. aureus extracts were respectively incubated with a rabbit antiserum (1:5000) raised against SrtA_{ΔN}. Bound antibody was detected with a goat anti-rabbit IgG alkaline phosphatase conjugate (1:20000). All protein bands were revealed after incubation of the membranes with the substrates 5-bromo-4-chloroindol-3-yl phosphate and Nitro Blue Tetrazolium.

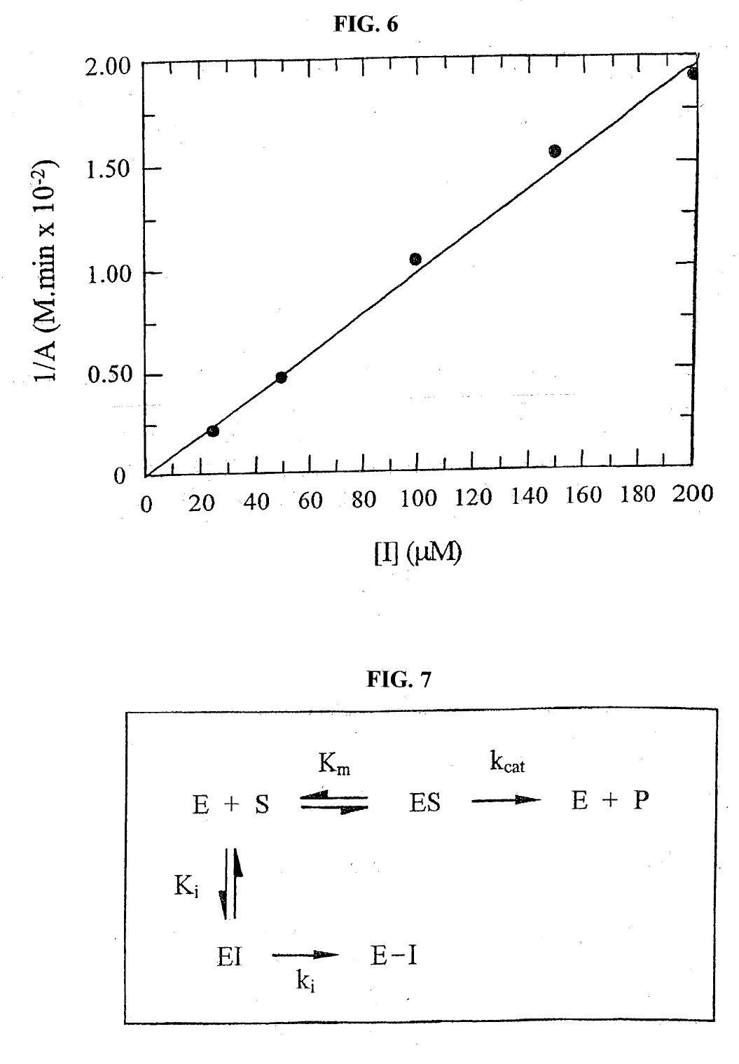

Progress curves for SrtA_{ΔN}-catalysed hydrolysis of the internally quenched substrate Dabcyl-Gln-Ala-Leu-Pro-Glu-ThrGly-Glu-Glu-Edans in the presence of different concentrations of Cbz-Leu-Pro-Ala-Thr-CHN₂ (I) and Cbz-Leu-Pro-Ala-ThrCH₂Cl (II) typified the action of active site-directed irreversible inhibitors operating via the mechanism illustrated in FIG. 7 [28,29]. Data from the curves were fitted, using non-linear regression analysis [29], to the integrated rate equation [P]=PJ1-e “-Pp”). This equation represents a first-order rate process for the formation of product P as a function of time, where kₐₚₚ is the apparent rate constant and P. represents the concentration of product at a time approaching infinity. Using five different concentrations for each inhibitor (25, 50, 100, 150 and 200/μM), the values kₐₚₚ and P_{∞}were determined and utilized to evaluate the apparent second-order rate constant A for the inactivation of SrtA_{ΔN} in the presence of the substrate [28]. The individual kinetic constants kᵢ and Kᵢ were then evaluated for both inhibitors from a plot of 1/A versus inhibitor concentration [I] [28], and the specificity constant k;/K; was calculated. FIG. 6 shows the plot for inactivation of SrtA_{ΔN} by the peptidyl-chloromethane inhibitor Cbz-Leu-Pro-Ala-Thr-CH,Cl (II). All the kinetic constants determined for inhibitors I and II are given in Table 1 below.

Table 1 Kinetic Constants for the Inactivation of SrtA_{oN} by the Substratederived Peptidyl-diazomethane (I) and Peptidyl-chloromethane (II) Inhibitors

Data from the progress curves for Srt_{ΔN}-catalysed hydrolysis of the internally quenched substrate Dabcyl-Gln-Ala-Leu-Pro-Glu-Thr-Gly-Glu-Glu-Edans in the presence of five different concentrations of inhibitors I and II (25, 50, 100, 150 and 200, W) were fitted by non-linear regression analysis to the integrated rate equation [P]=P_{∞}(1-e^{-kaPPt}). The P_{∞}. and kapp values were determined and used to evaluate the apparent second-order rate constant A for inactivation of Srt_{ΔN} in the presence of substrate. The individual kinetic constants k and K were then evaluated from a plot of 1/A versus inhibitor concentration [I], and the specificity constant for each inhibitor (kᵢKᵢ) calculated. Values represent the means ±S.E.M. for four determinations.

| Inhibitor | k₁ (min⁻¹) | Kᵢ (M) | kᵢ/Kᵢ (M⁻¹ − min⁻¹) |
|---|---|---|---|
| I | 5.8 ± 0.6 × 10⁻³ | 2.2 ± 0.2 × 10⁻⁷ | 2.2 ± 0.2 × 10⁴ |
| II | 1.1 ± 0.1 × 10⁻² | 2.1 ± 0.2 × 10⁻⁷ | 5.3 ± 0.6 × 10⁴ |

The first point of interest was the observation that the peptidylchloromethane inhibitor (II) inactivates SrtA_{ΔN} by approx. 2-fold more rapidly than the analogous diazomethane sequence (1), as revealed by the specificity constants kᵢ/Kᵢ=5.3×10⁴ and 2.2×10⁴ M⁻¹-min⁻¹ respectively. Both inhibitors I and 11 exhibited almost identical, sub-micromolar inhibitor constants Kᵢ=2.2×10⁻¹ and 2.1×10⁻¹ M respectively. This implies high affinity of interaction between the common tetrapeptide motif of each inhibitor and the SrtA_{ΔN} enzyme. The greater effectiveness of the chloromethane inhibitor (II) can be attributed almost exclusively to the increased rate at which it covalently modifies SrtA_{ΔN}. Therefore the peptidyl-diazomethane (I) covalently modifies SrtA_{ΔN} with a first-order rate constant kᵢ=5.8×10⁻³ min⁻¹, whereas the peptidyl-chloromethane (II) covalently modifies the enzyme by approx. 2-fold more rapidly (kᵢ=1.1×10⁻² min⁻¹). This observation fits the known relative chemical reactivity of the electrophilic groupings of both inhibitors towards thiol nucleophiles [27,30]. The first-order rate constants observed are considerably smaller than those previously determined for the inactivation of cysteine proteases by active-site directed peptidyl-diazomethanes and peptidyl-chloromethanes. For example, the peptidyl-chloromethane inhibitor Cbz-Phe-Phe-CH,Cl alkylates the active-site cysteine residue of cathepsin B with kᵢ=12.5 min⁻¹ [30]. This is approx. 1000-fold greater than the irreversible modification of SrtA_{ΔN} by the peptidyl-chloromethane sequence (II).

The potential of a biotinylated inhibitor of SrtA for the detection of functionally active forms of SrtA-like species in Gram-positive organisms was evaluated. Towards this end, the biotinylated inhibitor sequence biotin-Ahx-Leu-Pro-Ala-Thr-CHN₂ (III) was utilised. The strategy underlying the design of III was based on two tenets. First, the peptidyl-diazomethane (I) was chosen over the slightly more active chloromethane sequence (11), as the latter has potential to cause non-specific alkylation of biomolecules [27]. Secondly, an aminohexanoic spacer group was incorporated between the recognition motif of the inhibitor and the biotin moiety to maximize the interaction with streptavidin alkaline phosphatase, which was used for disclosure of affinitylabelled proteolytic species after SDS/PAGE and Western-blot analysis.

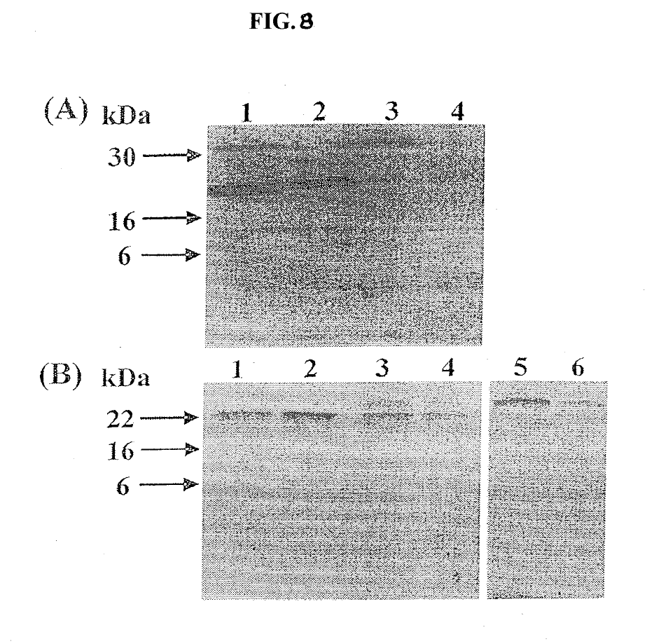

During initial Western-blotting experiments, biotin-Ahx-LeuPro-Ala-Thr CHN₂ (III) (50, μM) was examined for its ability to disclose SrtA_{ΔN} in E. coli cells transfected with the SrtA gene from S. aureus (within the pET-3d expression vector; see FIG. 5A). As shown in FIG. 8, lane 1 contained a crude lysate prepared from a freshly induced culture of the transfected E. coli cells, whereas lane 2 contained the ‘flow-through’ from the NiNTA-agarose column used to purify the expressed Hisₛ-tagged SrtA_{ΔN}. In lane 3, the bound fraction eluted from the column with 10 mM imidazole (purified SrtA_{ΔN}) was also analysed. In both lanes 1 and 2, the intensely stained protein species with an apparent molecular mass of 22 kDa was the endogenous biotin carbonyl carrier protein (BCCP). This species was totally absent in the fraction selectively eluted from the Ni-NTA-agarose column (FIG. 8, lane 3). The band with an apparent molecular mass of approx. 30 kDa, which was present in both the crude cell lysate (FIG. 8, lane 1) and eluted fraction (FIG. 8, lane 3), was His₆-tagged SrtA_{ΔN}. The identity of the affinity-labelled SrtA_{ΔN} band was confirmed with a monoclonal antibody to the His, sequence and a rabbit antiserum raised against the recombinant enzyme (results not shown). The molecular mass determined for SrtA_{ΔN} by SDS/PAGE was in agreement with the previous reports of Ton-That et al. [8]. In lane 4, pre-treatment of the purified Srt_{ΔN} sample with the thiol-directed inhibitor pHMB, before ‘probing’ with biotin-Ahx-Leu-Pro-Ala-ThrCHN₂ (III), resulted in complete diminution of SrtA_{ΔN} labelling. As the enzyme contains only a single cysteine residue (Cys¹⁸⁴) essential for catalytic activity [8,9], we can confidently conclude that the binding of this biotinylated inhibitor to SrtA_{ΔN} is indeed active-site-directed.

Western-blotting experiments were performed with biotinAhx-Leu-Pro-Ala-Thr-CHN₂ (III) to detect the presence of wildtype SrtA in crude cell lysates from S. aureus (Oxford strain; see FIG. 8B). In the duplicate control lanes 1 and 2, which contained unprobed cell lysate, the BCCP protein (22 kDa) was again observed. In the duplicate lanes 3 and 4, which contained cell lysate “probed” with biotin-Ahx-Leu-Pro-Ala-Thr-CHN₂ (III), the BCCP protein was detected along with an additional protein of apparent molecular mass 24 kDa. This molecular mass was consistent with the predicted value for wild-type SrtA. That this protein band was indeed affinity-labelled SrtA was confirmed with an antiserum raised against the recombinant form of the protein (see FIG. 8B).

The discrete labelling of only recombinant and wild-type forms of SrtA in crude cell lysates prepared from transfected E. coli cells and S. aureus respectively provides convincing evidence for the selectivity of action of the biotin-Ahx-Leu-Pro-Ala-Thr-CHN₂ inhibitor. As expected, biotin-Ahx-Leu-Pro-Ala-Thr-CHN₂ (III) selectively disclosed wild-type SrtA, but not SrtB in crude S. aureus extracts. Two aspects explain the specificity of our affinity label for SrtA only. First, SrtA is a constitutively expressed protease in S. aureus, whereas SrtB expression is tightly regulated by the concentration of iron, which was not added to our culture media [11]. Secondly, the previous studies of Mazmanian et al. [11] demonstrated the exquisite specificity of SrtA and SrtB towards cognate synthetic peptides modelled on their respective sorting signals. When tested against internally quenched fluorescent peptides, SrtB was shown only to cleave peptides containing the -Asn-Pro-Gln-Thr↓Asn-sorting sequence, and not the -Leu-Pro-Xaa-Thr↓Gly-sequence recognized by SrtA. In contrast, SrtA displayed exactly the contrary specificity. Consequently, we would not have expected SrtB to be inhibited by our biotinylated affinity label, even under conditions where the enzyme was expressed.

Biotinylated versions of the inhibitors of the invention may be used to detect additional sortase species. Identification of such species in gram positive organisms could lead to their validation as therapeutic targets. The inhibitors of the invention may also be used to block bacterial adherence in animals and humans and thus limit infection. When used in combination with other therapeutic regimes, they could lead to more rapid clearing of infection. The inhibitors could also be used in combination with vaccine approaches to prevent the display of decoy or cloaking proteins, or be used as adjuvant therapies in limiting bacterial infection in burns patients, by blocking adherence to exposed sub-epithelial tissue. The inhibitors could also be used to reduce bacterial adherence to medical devices such as stents, catheters and prosthesis.

For pharmaceutical use, the inhibitor may be combined with a pharmaceutically acceptable carrier to form a pharmaceutical composition. Pharmaceutically acceptable carriers include physiologically tolerable or acceptable diluents, excipients, solvents, adjuvants, or vehicles, for parenteral injection, for intranasal or sublingual delivery, for oral administration, for rectal or topical administration or the like.

For oral administration, the medicament according to the invention may be in the form of, for example, a tablet, capsule suspension or liquid. The medicament is preferably made in the form of a dosage unit containing a particular amount of the active ingredient. Examples of such dosage units are capsules, tablets, powders, granules or a suspension, with conventional additives such as lactose, mannitol, corn starch or potatoes starch; with binders such as crystalline cellulose, cellulose derivatives, acacia, corn starch or gelatins; with disintegrators such as corn starch, potaote starch or sodium carboxymethyl-cellulose; and with lubricants such as talc or magnesium stearate. The active ingredient may also be administered by injection as a composition wherein, for example, saline, dextrose or water may be used as a suitable carrier.

For intravenous, intramuscular, subcutaneous, or intraperitioneal administration, the compound may be combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering said solution sterile. The formulations may be present in unit or multi-dose containers such as seated ampoules or vials.

If the disease or condition to be treated is localized in the G.I. tract, the compound may be formulated with acid-stable, base-liable coatings known in the art which began to dissolve in the high pH intestine. Formulations to enhance local pharmacologic effects and reduce systemic uptake are preferred.

Formulations suitable for administration conveniently comprise a sterile aqueous preparation of the active compound which is preferably made isotonic. Preparations for injections may also be formulated by suspending or emulsifying the compounds in non-aqueous solvent, such as vegetable oil, synthetic aliphatic acid glycerides, esters of higher aliphatic acids or propylene glycol.

Formulations for topical use include known gels, creams, oils, and the like. For aerosol delivery, the compounds may be formulated with known aerosol exipients, such as saline and administered using commercially available nebulizers. Formulation in a fatty acid source may be used to enhance biocompatibility.

For rectal administration, the active ingredient may be formulated into suppositories using bases which are solid at room temperature and melt and dissolve at body temperature. Commonly used bases include cocoa butter, glycerinated gelatin, hydrogenated vegetable oil, polyethylene glycols of various molecular weights, and fatty esters of polyethylene stearate.

The dosage form and amount can be readily established by reference to known anti-bacterial or vaccination treatment or prophylactic regiments. The amount of therapeutically active compound that is administered and the dosage regimen for treating a disease condition with the compounds and/or compositions of this invention depends on a variety of factors, including the age, weight, sex and medical condition of the subject, the severity of the disease, the route and frequency of administration, and the particular compound employed, the location of the disease or condition, as well as the pharmacokinetic properties of the individual treated, and thus may vary widely. The dosage will generally be lower if the compounds are administered locally rather than systemically, and for prevention rather than for treatment. Such treatments may be administered as often as necessary and for the period of time judged necessary by the treating physician. One of skill in the art will appreciate that the dosage regime or therapeutically effective amount of the inhibitor to be administrated may need to be optimized for each individual. The pharmaceutical compositions may contain active ingredient in the range of about 0.1 to 2000 mg, preferably in the range of about 0.5 to 500 mg and most preferably between about 1 and 200 mg. A daily dose of about 0.01 to 100 mg/kg body weight, preferably between about 0.1 and about 50 mg/kg body weight, may be appropriate. The daily dose can be administered in one to four doses per day.

The invention is not limited to the embodiments hereinbefore described which may be varied in construction and detail without departing from the spirit of the invention.

References

1. Navarre, W. W. and Schneewind, 0. (1999) Surface proteins of Gram-positive bacteria and mechanisms of their targeting to the cell wall envelope. Microbiol. Mol. Biol. Rev. 63, 174-229

2. Flock, J. I. (1999) Extracellular-matrix binding proteins as targets for the prevention of Staphylococcus aureus infections. Mol. Med. Today 5, 532-537

3. Schneewind, 0., Model, P. and Fischetti, V. A. (1992) Sorting of protein A to the staphylococcal cell wall. Cell (Cambridge, Mass.) 70, 267-281

4. Schneewind, 0., Mihaylova-Petkov, D. and Model, P. (1993) Cell wall sorting signals in surface proteins of Gram-positive bacteria. EMBO J. 12, 4803-4811

5. Navarre, W. W. and Schneewind, 0. (1994) Proteolytic cleavage and cell wall anchoring at the LPXTG motif of surface proteins in Gram-positive bacteria. Mol. Microbiol. 14, 115-121

6. Mazmanian, S. K., Liu, G., Ton-That, H. and Schneewind, 0. (1999) Staphylococcus aureus sortase, an enzyme that anchors surface proteins to the cell wall.Science 285, 760-763

7. Ton-That, H. and Schneewind, 0. (1999) Anchor structure of staphylococcal surface proteins. IV. Inhibitors of the cell wall sorting reaction. J. Biol. Chem. 274, 24316-24320

8. Ton-That, H., Liu, G., Mazmanian, S. K., Faull, K. F. and Schneewind, 0. (1999) Purification and characterisation of sortase, the transpeptidase that cleaves surface proteins of Staphylococcus aureus at the LPXTG motif. Proc. Natl. Acad. Sci. U.S.A. 96,12424-12429

9. Ton-That, H., Mazmanian, S. K., Faull, K. F. and Schneewind, 0. (2000) Anchoring of surface proteins to the cell wall of Staphylococcus aureus. I. Sortase catalysed in vitro transpeptidation reaction using LPXTG peptide and NH_{z} GIy₃ substrates. J. Biol. Chem. 275, 9876-9881

10. Ton-That, H., Faull, K. F. and Schneewind, 0. (1997) Anchor structure of staphylococcal surface proteins. I. A branched peptide that links the carboxyl terminus of proteins to the cell wall. J. Biol. Chem. 272, 22285-22292

11. Mazmanian, S. K., Ton-That, H., Su, K. and Schneewind, 0. (2002) An iron-regulated sortase anchors a class of surface protein during Staphylococcus aureus pathogenesis. Proc. Natl. Acad. Sci. U.S.A. 99, 2293-2298

12. Mazmanian, S. K., Liu, G., Jensen, E. R., Lenoy, E. and Schneewind, 0. (2000) Staphylococcus aureus sortase mutants defective in the display of surface proteins and in the pathogenesis of animal infections. Proc. Natl. Acad. Sci. U.S.A. 97, 5510-5515

13. Bolken, T. C., Franke, C. A., Jones, K. F., Zeller, G. 0., Jones, C. H., Dutton, E. K. and Hruby, D. E. (2001) Inactivation of the srtA gene in Streptococcus gordonii inhibits cell wall anchoring of surface proteins and decreases in vitro and in vivo adhesion Infect. Immun. 69, 75-80

14. Doyle, M. P.; McKervey, M. A. (1997) Chem Commun. 11, 983-989

15. Miller, D. J.; Moody, C. J. (1995) Tetrahedron 51, 10811-10843

16. Schleidt, K. A.; Roush, W. R.; McKerrow, J. H.; Selzer, P. M.; Hansell, E.; Rosenthal, P. J. (1998) Bioorg. Med. Chem. 6, 2477-2494

17. Hanzlik, R. P.; Thompson, S. A.(1984) J. Med. Chem. 38, 3193-3196

18. Walker, B. (1994) Solid Phase Peptide Synthesis. In: Peptide Antigens, A Practical Approach (Wisdom, G. B.,ed.) pp. 27-81, IRL Press, Oxford

19. Walker, B., Cullen, B. M., Kay, G., Halliday, I. M., McGinty, A. and Nelson, J. (1992) The synthesis, kinetic characterization and application of a novel biotinylated affinity label for cathepsin B. Biochem. J. 283, 449-453

20. Lynas, J. F., Hawthorne, S. J. and Walker, B. (2000) Development of peptidyl α-keto-β-aldehydes as new inhibitors of cathepsin L-comparisons of potency and selectivity profiles with cathepsin B. Bioorg. Med. Chem. Lett. 10, 1771-1773

21. Walker, B., Lynas, J. F., Meighan, M. A. and Bromme, D. (2000) Evaluation of dipeptide a-keto-(l-aldehydes as new inhibitors of cathepsin S. Biochem. Biophys. Res. Commun. 275, 401-405

22. Walker, B. (1994) in Synthetic Antigens: A Practical Approach (Wisdom, G. B., ed.), pp. 55-99, Oxford University Press, Oxford

23. Green, G. D. J. and Shaw, E. (1981) Peptidyl diazomethyl ketones are specific inactivators of thiol proteinases. J. Biol. Chem. 256, 1923-1928

24. Cullen, B. M., Halliday, I. M., Kay, G., Nelson, J. and Walker, B. (1992) The application of a novel biotinylated affinity label for the detection of a cathepsin B-like precursor produced by breast tumour cells in culture. Biochem. J. 283, 461-465

25. McGinty, A., Moore, M., Halton, D. W. and Walker, B. (1993) Characterisation of the cysteine proteinases of the common liver fluke Fasciola Hepatica using novel, active-site directed affinity labels. Parasitology 106, 487-493

26. Hawthorne, S. J., Pagano, M., Harriott, P., Halton, D. W. and Walker, B. (1998) The synthesis and utilization of 2,4-dinitrophenyl-labeled irreversible peptidyl diazomethyl ketone inhibitors. Anal. Biochem. 261, 131-138

27. Shaw, E. (1990) Cysteinyl proteinases and their selective inactivation. Adv. Enzymol. Relat. Areas Mol. Biol. 63, 271-347

28. Tian, W. X. and Tsou, C. L. (1982) Determination of the rate constant of enzyme modification by measuring the substrate reaction in the presence of the modifier. Biochemistry 21, 1028-1032

29. Walker, B. and Elmore, D. T. (1984) The irreversible inhibition of urokinase, kidney cell plasminogen activator, plasmin and (l-trypsin by 1-(M6-amino-n-hexyl)-carbamoyl imidazole. Biochem. J. 221, 277-280

30. Rauber, P., Angliker, H., Walker, B. and Shaw, E. (1986) The synthesis of peptidylfluoromethanes and their properties as inhibitors of serine proteinases and cysteine proteinases. Biochem. J. 239, 633-640

31. Duffy, J. M., Walker, B., Guthrie, D., Grimshaw, J., McNally, G., Grimshaw, J. T., Spedding, P. L. and Mollan, R. A. B. (1994) The detection, quantification and partial characterisation of cathepsin B-like activity in human pathological synovial fluids. Eur. J. Clin. Chem. Clin. Biochem. 32, 429-434

1. A modified peptide comprising the formula: 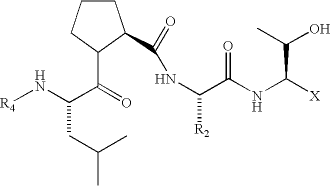
wherein R₂ is a side chain of any amino acid, R₄ is an acetyl group or an N-protecting group, and wherein X is an electrophilic group having an ability to undergo nucleophilic attack with active site cysteine residues in cysteine proteinase enzymes.

2. The modified peptide as claimed in claim 1 in which X is a substituent selected from the group consisting of: 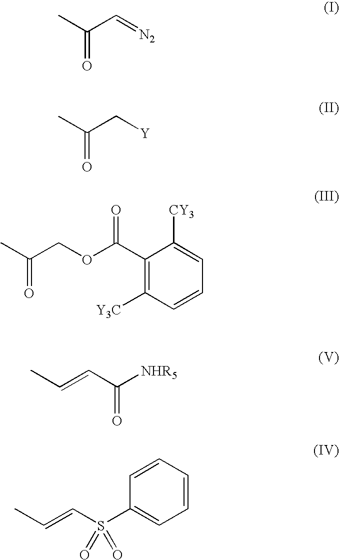
wherein Y is a halogen and R₅ is a natural amino acid side chain.

3. The modified peptide as claimed in claim 1 including an assayable label.

4. The modified peptide as claimed in claim 1 in which the N-protecting group comprises an assayable label linked to a spacer group.

5. The modified peptide as claimed in claim 4 in which the spacer group is an aminohexanoyl group.

6. The modified peptide as claimed in claim 4 in which R₄ is biotin-bis-aminohexanoic acid.

7. The modified peptide as claimed in claim 1 in which the N-protecting group is a benzyloxycarbonyl group (Cbz).

8. The modified peptide as claimed in claim 1 in which R₂ is a side chain of alanine.

9. The modified peptide according to claim 2 in which in substituent III the halogen is fluorine.

10. The modified peptide as claimed in claim 2 in which in substituent II the halogen is chlorine.

11. The modified peptide having the molecular structure (I) of FIG. 4.

12. The modified peptide having the molecular structure (II) of FIG. 4.

13. The modified peptide having the molecular structure (III) of FIG. 4.

14. A method for inhibiting the activity of a cysteine peptidase enzyme comprising contacting the enzyme with a modified peptide according to claim 1.

15. The method according to claim 14 wherein the cysteine peptidase enzyme inhibited is a sortase enzyme.

16. The method according to claim 14 wherein the cysteine peptidase enzyme inhibited is staphylococal surface protein sorting A (SrtA).

17. A medicament comprising a modified peptide according to claim 1.

18. A medicament comprising a modified peptide having a molecular structure selected from the group of molecular structures (I), (II) and (III) of FIG. 4.

19. A method of treating a bacterial infection in an individual comprising the step administering to the individual an effective amount of a modified peptide according to claim 1.

20. A method for detecting the presence of a functionally active serine protease enzyme in a material comprising contacting the material with a modified peptide according to claim 1.

21. The method according to claim 20 wherein the functionally active serine protease enzyme detected is a sortase enzyme.

22. The method according to claim 20 wherein the functionally active serine protease enzyme detected is SrtA or SrtA-like enzyme.

23. A method of purifying functionally active SrtA or SrtA-like enzymes in a biological extract, which method comprises the steps of:
incubating the biological extract with a labelled modified peptide according to claim 2 to allow the labelled modified peptide bind to any SrtA or SrtA-like enzymes present in the extract; and
purifying any thus labelled enzymes.

24. A method as claimed in claim 23 in which the purification step comprises affinity chromatography.

25. A method as claimed in claim 24 in which the label is biotin, and in which the chromatography column comprises streptavidin.

26. A method as claimed in claims 23 including one or more of the further steps of mass spectral analysis and amino acid sequencing of the purified enzyme.

27. A method of detecting functionally active SrtA or SrtA-like enzymes in a biological extract, which method comprises the steps of:
incubating the biological extract with a labelled modified peptide according to claim 3 to allow the labelled modified peptide bind to the or each SrtA or SrtA-like enzyme; and
treating the incubated biological extract to separate out proteins in the extract; and
carrying out a Western-blot analysis of the separated proteins.

28. A method as claimed in claim 27 in which the proteins in the extract are separated using polyacrylamide gel electrophoresis.

29. A pharmaceutical composition comprising a pharmaceutically acceptable carrier and a modified peptide according to claim 1.

30. A pharmaceutical composition comprising a pharmaceutically acceptable carrier and a modified peptide having a molecular structure selected from the group of molecular structures consisting of (I), (II) and (III) of FIG. 4.
